(19) United States
(12) Patent Application Publication
(10) Pub. No.: US 2013/0018360 A1
(43) Pub. Date: Jan. 17, 2013

(54) METHOD FOR DELIVERING OPHTHALMIC DRUGS

(71) Applicants: Marissa Dockendorf, Thousand Oaks, CA (US); Günter Solms, Jacksonville Beach, FL (US); Bailin Laing, St. Johns, FL (US); Bret A. Coldren, Vista, CA (US); Gary Yewey, Jacksonville, FL (US); Gerald Yewey, Jacksonville, FL (US)

(72) Inventors: Marissa Dockendorf, Thousand Oaks, CA (US); Günter Solms, Jacksonville Beach, FL (US); Bailin Laing, St. Johns, FL (US); Bret A. Coldren, Vista, CA (US); Gary Yewey, Jacksonville, FL (US); Gerald Yewey, Jacksonville, FL (US)

(21) Appl. No.: 13/530,317

(22) Filed: Jun. 22, 2012

Related U.S. Application Data
(60) Provisional application No. 61/507,154, filed on Jul. 13, 2011.

(51) Int. Cl. A61F 9/00

(52) U.S. Cl. 604/892.1; 604/290

(57) ABSTRACT

This invention relates to a method of delivering ophthalmic drugs, specifically prostaglandins and prostamides. This method may also be applied to other types of intraocular pressure (IOP)-lowering drugs (e.g. carbonic anhydrase inhibitors, beta blockers, alpha-adrenergic agonists, and parasympathomimetics) as well as other types of ophthalmic drugs for other indications (e.g. dry eye, inflammation, and infection).

### CROSS REFERENCE TO RELATED APPLICATIONS

This application relates to U.S. patent application Ser. No. 61/507,154, filed Jul. 13, 2011; all applications are herein incorporated by reference in their entireties.

### FIELD OF THE INVENTION

This invention relates to a method of delivering ophthalmic drugs, specifically prostaglandins and prostamides, preferably via ocular drug delivery devices that may be inserted into the lacrimal punctum, such as an external precision doser/dropper, or an ocular implant. This method may also be applied to other types of intraocular pressure (IOP)-lowering drugs (e.g. carbonic anhydrase inhibitors, beta blockers, alpha-adrenergic agonists, and parasympathomimetics) as well as other types of ophthalmic drugs for other indications (e.g. dry eye, inflammation, and infection).

### BACKGROUND OF THE INVENTION

Active agents frequently are administered to the eye for the treatment of ocular diseases and disorders. Conventional means for delivering active agents to the eye involve topical application to the surface of the eye. The eye is uniquely suited to topical administration because when properly constituted, topically applied active agents can penetrate through the cornea and rise to therapeutic concentration levels inside the eye. Active agents for ocular diseases and disorders may be administered orally or by injection, but such administration routes are disadvantageous in that, in oral administration, the active agent may reach the eye in too low a concentration to have the desired pharmacological effect and their use is complicated by significant, systemic side effects and injections pose the risk of infection.

The majority of ocular active agents are currently delivered topically using eye drops which, though effective for some applications, are inefficient. When a drop of liquid is added to the eye, it overfills the conjunctival sac, the pocket between the eye and the lids, causing a substantial portion of the drop to be lost due to overflow of the lid margin onto the cheek. In addition, a substantial portion of the drop that remains on the ocular surface is drained into the lacrimal puncta, diluting the concentration of the drug.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision, and pediatric and psychiatric patient populations pose difficulties as well.

It is known to use devices that may be inserted into one or more of an orifice of an individual's eye, such as a lacrimal punctum, to deliver active agents. One disadvantage of using such devices to deliver agents is that much of the agent may delivered in an initial, large bolus upon insertion of the device into the eye rather than a more linear delivery of the agent over time.

Prior topical sustained release systems include gradual release formulations, either in solution or ointment form, which are applied to the eye in the same manner as eye drops but less frequently. Such formulations are disclosed, for example, in U.S. Pat. No. 3,826,258 issued to Abraham and U.S. Pat. No. 4,923,699 issued to Kaufman. Due to their method of application, however, these formulations result in many of the same problems detailed above for conventional eye drops. In the case of ointment preparations, additional problems are encountered such as a blurring effect on vision and the discomfort of the sticky sensation caused by the thick ointment base.

Alternatively, sustained release systems have been configured to be placed into the conjunctival sac, between the lower lid and the eye. Such units typically contain a core drug-containing reservoir surrounded by a hydrophobic copolymer membrane which controls the diffusion of the drug. Examples of such devices are disclosed in U.S. Pat. No. 3,618,604 issued to Ness, U.S. Pat. No. 3,626,940 issued to Zaffaroni, U.S. Pat. No. 3,845,770 issued to Theeuwes et al., U.S. Pat. No. 3,962,414 issued to Michaels, U.S. Pat. No. 3,993,071 issued to Higuchi et al., and U.S. Pat. No. 4,014,335 issued to Arnold. However, due to their positioning, the units are uncomfortable and poor patient acceptance is again encountered.

It would be desirable, therefore, to be able to deliver very small quantities of a therapeutic agent to the eye, without the drawbacks or present methods, such as wasted medication due to overfilling of the conjunctival sac, or the use of large quantities of medication to reach therapeutic compliance.

### BRIEF DESCRIPTION OF THE DRAWINGS

FIG. 1 is an illustrative embodiment of a punctal plug device with a porous-matrix drug core containing therapeutic agent formulation.

FIG. 2 is an axial cross-section of the device of FIG. 1.

FIG. 3 is an illustrative depiction of a punctal plug device according to an embodiment of the present invention that is shown comprising a drug core with a plurality of channels defined by a septum material.

FIG. 4 is an expanded cross-sectional view of an exemplary drug core according to the present invention, shown comprising contiguous porous matrix material infused with a contiguous therapeutic agent formulation.

FIG. 5 is a cross-sectional view of FIG. 3 showing the drug core in combination with a plurality of channels defined by a septum material and enclosed in the outer body of the punctal plug device.

FIG. 6 is a cross-sectional view of FIG. 3 showing the drug core in combination with four channels defined by a septum material, enclosed in the outer body of the punctal plug device.

### DETAILED DESCRIPTION OF ILLUSTRATIVE EMBODIMENTS OF THE INVENTION

This invention relates generally to methods for delivering ophthalmic drugs, specifically prostaglandins and prostamides, preferably via ocular drug delivery devices inserted into the lacrimal punctum. This method may also be applied to other types of intraocular pressure (IOP)-lowering drugs (e.g. carbonic anhydrase inhibitors, beta blockers, alpha-adrenergic agonists, and parasympathomimetics) as well as other types of ophthalmic drugs for other indications (e.g. dry eye, inflammation, and infection).

Typically, ophthalmic drugs are administered topically in volumes of 30-70 μL. It has been unexpectedly found, however, that similar or better efficacy may be achieved by topically administering smaller (e.g., nanoliter sized) doses of concentrated drug (i.e. of higher drug concentration than the commercially available ophthalmic drug formulation). In one illustrative embodiment of the invention, topical doses of concentrated drug may be applied in volumes of 40 pL to 100 nL every 12 to 36 hours such that the total amount of drug applied is equivalent to or 10-fold less than the amount that is contained within a commercial ophthalmic drop. Depending on the specific active-agent containing therapeutic agent used, the doses may be in the range of from about 1 pL to about 1000 nL and the dosing may take place about every 1 to about every 72 hours.

For example, there are about 9 μg of bimatoprost in a 30 μL Lumigan (0.03% bimatoprost) dose. In embodiments of the present invention similar efficacy can be attained by applying 0.9 to 9 μg of bimatoprost using a concentrated formulation (>0.03% bimatoprost) and a much smaller dose volume (40 pL to 100 nL). The concentrated formulation may be achieved by mixing the active ingredient (e.g. bimatoprost, latanoprost, or travoprost) with one or more excipients. Exemplary of such excipients are propylene glycol, castor oil, and/or ethyl oleate.

Delivery of ophthalmic drugs via topical application (30-70 μL) results in drug wastage and potential adverse effects. Only ˜1-10% of the drug delivered via prior art meant is actually absorbed by the eye. The remainder of the drug either runs down the patient's cheek or is drained through the nasolacrimal system, where it is available for systemic absorption and can potentially result in unwanted side effects.

Delivering the drug in nanoliter quantities would reduce this drug wastage. Furthermore, some patients are noncompliant with administering ophthalmic drops as recommended, either not taking their dose at the recommended time or frequency or incorrectly applying their drops. There have been attempts to develop extended ophthalmic drug delivery devices (such as punctal plugs, ocular inserts, contact lenses, etc.); however, sustained delivery of low doses (i.e. doses less than that typically applied in the commercially approved drop) has not yet been proven to be efficacious in comparison to using drops.

Furthermore, in instances where the pharmacology of the drug may not be supportive of continuous application of the drug (e.g. tolerance to prostaglandins and prostamides may develop), pulsatile delivery of the therapeutic agent may indeed be preferable to a low-order (or zero-order) continuous delivery profile. Additionally, capacity limitations of these devices become a concern when attempting to achieve delivery for long durations of time. Delivery of dosages in nL quantities in a pulsatile manner (e.g. every 12-36 hrs) through a delivery device (e.g MEMS pump) could maximize drug payload and minimize packaging size of the drug reservoir, and allow for longer duration of device efficacy. Furthermore, such devices could deliver the drug in a pulsatile manner, thus avoiding potential tolerance to the drug.

Turning now to the drawing figure, punctal plugs are an exemplary means of achieving the dosing objective of the present invention. Punctal plugs have been in use for decades now to treat conditions of dry eye. More recently they have gained attention for use as drug delivery systems for the treatment of ocular diseases and conditions. Several challenges exist with formulating a drug to release at the desired daily rate and or dose that will give efficacy while limiting adverse events. Disclosed are numerous embodiments of punctal plugs which may be employed in embodiments of the present invention.

Diffusion based drug delivery systems are characterized by release rate of drug is dependent on its diffusion through inert water insoluble membrane barrier. There are basically two diffusion designs: reservoir devices and matrix devices. Reservoir devices are those in which a core of drug is surrounded by polymeric membrane. The nature of the membrane determines the rate of release of drug from system. The process of diffusion is generally described by a series of equations governed by Fick's first law of diffusion. A matrix device consists of drug dispersed homogenously throughout a polymer.

Reservoir and matrix drug delivery systems are considered diffusion based sustained release systems and constitute any dosage form that provides medication over an extended period of time. The goal of a sustained release system is to maintain therapeutic levels of drug for an extended period and this is usually accomplished by attempting to obtain zero-order release from the sustained release system. Sustained release systems generally do not attain this type of release profile but try to approximate it by releasing in a slow first order manner. Over time, the drug release rate from reservoir and matrix sustained release systems will decay and become non therapeutic.

Zero-order drug release constitutes drug release from a drug delivery system at a steady sustained drug release rate, that is, the amount of drug that is released from the drug delivery system over equal time intervals does not decay and remains at the therapeutic level. This “steady sustained release drug delivery system” is referred to as a zero-order drug delivery system and has the potential to provide actual therapeutic control by its controlled release.

Another drug release profile is referred to as pulsatile drug delivery. Pulsatile drug delivery is intended to release a therapeutic amount of a therapeutic agent at regular intervals, thereby mimicking the episodic nature of “eye-drops”. Heretofore, pulsatile delivery drug cores for lacrimal devices have been limited by the complex nature of the drug-core structure and formulation and the small size of the lacrimal inserts, which are typically in the 3-5 mm range, but may be smaller or larger as particular sizes may be necessary to provide devices suitable for a large population where the size, since the size of the lacrimal canaliculus varies from person to person.

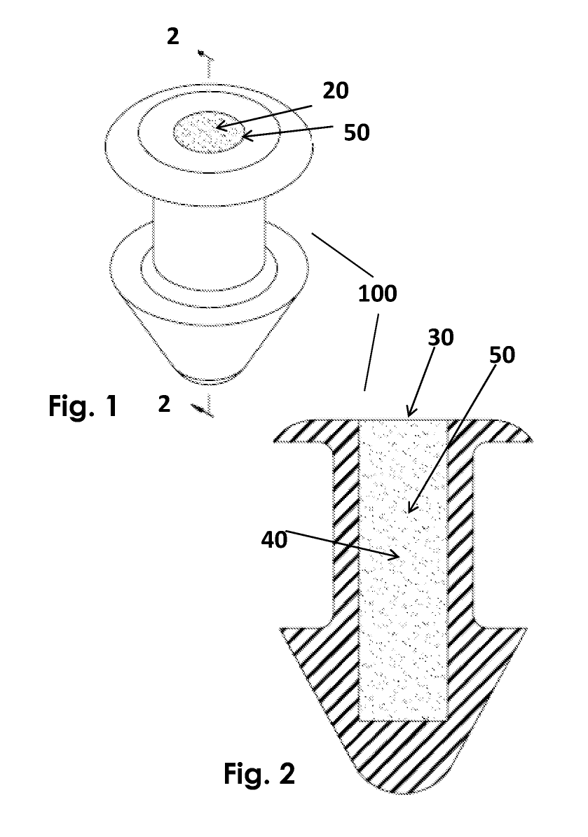

With specific reference now to the drawing figures, which are meant to be instructive, but not exhaustive of the possible structure and materials of the embodiments of the illustrative delivery devices for use with the present invention and wherein similar reference numerals refer to similar structure, an exemplary device having a drug-core configured release of a therapeutic agent is shown in FIG. 1.

As used herein, the term “active agent” refers to an agent capable of treating, inhibiting, or preventing a disorder or a disease. Exemplary active agents include, without limitation, pharmaceuticals and nutraceuticals. Preferred active agents are capable of treating, inhibiting, or preventing a disorder or a disease of one or more of the eye, nose and throat.

As used herein, the term “punctal plug” refers to a device of a size and shape suitable for insertion into the inferior or superior lacrimal canaliculus of the eye through, respectively, the inferior or superior lacrimal puncta. Exemplary and illustrative devices are disclosed in U.S. Pat. No. 6,196,993 and U.S. Published Patent Application No. 20090306608A1, both of which are hereby incorporated by reference in their entireties.

As used herein, the term “opening” refers to an opening in the body of a device of the invention of a size and shape through which the active agent can pass. Preferably, only the active agent and formulation can pass through the opening. The opening may be covered with a membrane, single or multiple pores, mesh, grid or it may be uncovered. The membrane, mesh, or grid may be one or more of porous, semi-porous, permeable, semi-permeable, and biodegradable.

An embodiment of the present invention may be described illustratively as having a structure that includes a pre-formed porous matrix material having a co-continuous microstructure, such that when said matrix is infused with a separately pre-formed drug formulation material, both materials remain distinct phases and retain continuity throughout themselves (as opposed to a co-formed solid solution or microencapsulation of solid dispersion). The porous-matrix-forming material is substantially immiscible with and not-solvated by the drug formulation. Drug solvation and controlled release is primarily via limited diffusion along the tortuous pores of the drug formulation phase, versus through the solid wall materials. The porous-matrix material can comprise a wetting agent to facilitate the wicking/loading of drug formulation into the pores.

In this illustrative embodiment of the present invention, the solid structure accommodates and stably-retains (i.e., is not easily squeezed out like a bulk liquid) a liquid drug formulation at a high mass fraction (>30%) while maintaining solid-like bulk properties such as having mechanical modulus significantly higher than the drug formulation itself, and comparable or higher than the plug body material. Such properties may be beneficial to punctum-insertion of the device because the device may otherwise have an unsuitably low consistency

FIG. 1 shows an illustrative lacrimal insert, or punctal plug 100. The plug 100 has a drug core insert 20. The structure of the plug is more fully described above and in the incorporated references is used by inserting it into the lacrimal canaliculus of the eye.

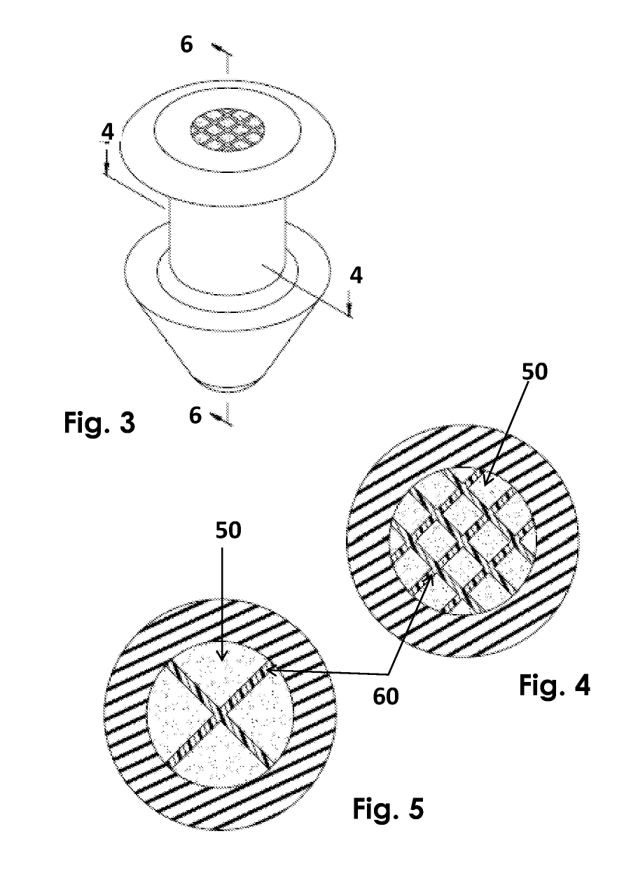

The device of FIG. 1 includes the drug core 20 that comprises a porous matrix material 50, further illustrated in FIG. 3, and more broadly shown in FIG. 2. In the cross-sectional view of FIG. 2, the porous drug matrix 40 may be comprised of, consist of, or consist essentially of a therapeutic agent formulation 50. The therapeutic agent formation 50 may be liquid, solid, gel or variants thereof and comprise, consist, or consist essentially of the drug formulations described hereinafter.

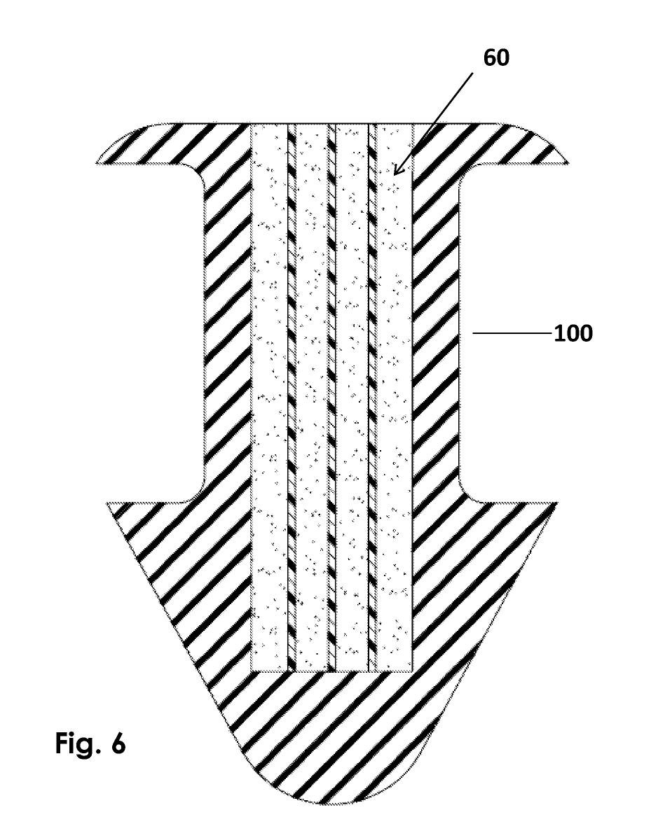

A barrier layer 30 may be included to retain the drug formulation within the punctal plug 100 and inhibit the elusion to the drug core and/or the therapeutic agent via the body of the punctal plug 100. Within the core itself, as shown in illustrative embodiments in FIGS. 4-6, there may be channels created by the placing septum material 60 in a matrixed arrangement within the core and then filling the matrixed arrangement with the drug core porous material, therapeutic agent, combinations thereof, etc. As illustrated, the drug core may be separated into a plurality of channels by the septum material 60. The desired drug release profile and mechanics of the drug core material may influence the number of channels.

The devices of the invention have a reservoir in which is found an active agent-containing material and an active agent therein. The active agent may be dispersed throughout the active agent-containing material or dissolved within the material. Alternatively, the active agent may be contained in inclusions, particulates, droplets, or micro-encapsulated within the material. Still as another alternative, the active agent may be covalently bonded to the material and released by hydrolysis, enzymatic degradation and the like. Yet as another alternative, the active agent may be in a reservoir within the material. While this may require the use of infusion apparatus different from that disclosed herein, those skilled in the art will recognize the manner of employing the active agent within the porous matrix or channels of the inventions described herein.

It is a discovery of the invention that the active agent may be released in a controlled manner, meaning over a period of time by using an active agent-containing material in which the agent is present in a substantially continuous concentration gradient throughout the material or by using a discontinuous concentration gradient. This is in contrast to a device that exhibits a therapeutically significant “burst” or immediate release upon insertion of an amount of active agent that is greater than the average release rate over time.

Without being bound to any particular theory, it is believed that an active agent-containing material that does not undergo significant chemical degradation during the time desired for the release of active agent will release the agent by diffusion through the matrix to a device's release surfaces, meaning surfaces of the active agent-containing material in contact with a person's body fluid. According to Fick's Law, the diffusive transport or flux, J, of the agent through the active agent-containing material is governed at each point and each time by the local concentration gradient, the diffusivity of the active agent with the material D, and the spatial variation of the cross-sectional geometry of the device.

Additionally, the spatial variation of the material's cross-sectional geometry may be used to control diffusivity. For example, if the material was in the form of a straight rod that has a uniform concentration of active agent, diffusivity will be reduced when the area at the open end of the material is significantly smaller than the average of the entire material. Preferably, the material area at the open end of the device is no more than one-half of the average cross sectional area of the material, meaning the cross section determined perpendicular to the primary dimension of active agent transport use.

One of ordinary skill in the art will recognize that, depending on how one varies one or more of the local concentration gradient, the diffusivity of the active agent from the material, and the spatial variation of the cross-sectional geometry of the device, a variety of release profiles may be obtained including, without limitation first order, second order, biphasic, pulsatile and the like. For example, either or both of the active agent concentration and diffusivity may increase from the surface to the center of the active agent-containing material in order to achieve more initial release. Alternatively, either or both may be increased or decreased and then increased again within the material to achieve a pulsatile release profile. The ability to achieve a variety of release profiles by varying local concentration gradient, the diffusivity of the active agent, and the spatial variation of the cross-sectional geometry may eliminate the need for rate-limiting membranes in the device.

Suitable polymeric materials for the active agent-containing material include, without limitation, hydrophobic and hydrophilic absorbable and non-absorbable polymers. Generally, liqua-gel and other soluble drug formulations are preferred. Alternatively, suitable hydrophobic, non-absorbable polymers include, without limitation, ethylene vinyl alcohol (“EVA”), fluorinated polymers including without limitation, polytetrafluoroethylene (“PTFE”) and polyvinylidene fluoride (“PVDF”), polypropylene, polyethylene, polyisobutylene, nylon, polyurethanes, polyacrylates and methacrylates, polyvinyl palmitate, polyvinyl stearates, polyvinyl myristate, cyanoacrylates, epoxies, silicones, copolymers thereof with hydrophobic or hydrophilic monomers, and blends thereof with hydrophilic or hydrophobic polymers and excipients.

Hydrophilic, non-absorbable polymers useful in the invention include, without limitation, cross-linked poly(ethylene glycol), poly(ethylene oxide), polypropylene glycol), poly(vinyl alcohol), poly(hydroxyethyl acrylate or methacrylate), poly(vinylpyrrolidone), polyacrylic acid, poly(ethyloxazoline), and poly(dimethyl acrylamide), copolymers thereof with hydrophobic or hydrophilic monomers, and blends thereof with hydrophilic or hydrophobic polymers and excipients.

Hydrophobic, absorbable polymers that may be used include, without limitation, aliphatic polyesters, polyesters derived from fatty acids, poly(amino acids), poly(ether-esters), poly(ester amides), polyalkylene oxalates, polyamides, poly(iminocarbonates), polycarbonates, polyorthoesters, polyoxaesters, polyamidoesters, polyoxaesters containing amine groups, phosphoesters, poly)anhydrides), polypropylene fumarates, polyphosphazenes, and blends thereof. Examples of useful hydrophilic, absorbable polymers include, without limitation, polysaccharides and carbohydrates including, without limitation, crosslinked alginate, hyaluronic acid, dextran, pectin, hydroxyethyl cellulose, hydroxy propyl cellulose, gellan gum, guar gum, keratin sulfate, chondroitin sulfate, dermatan sulfate, proteins including, without limitation, collagen, gelatin, fibrin, albumin and ovalbumin, and phospholipids including, without limitation, phosphoryl choline derivatives and polysulfobetains.

In one possible embodiment, the active agent-containing material is a polymeric material that is polycaprolactone. In still another, the material is poly(epsilon-caprolactone), and ethylene vinyl acetate of molecular weights between about 10,000 and 80,0000. About 0 to about 100 weight percent polycaprolactone and about 100 to about 0 weight percent of the ethylene vinyl acetate are used based on the total weight of the polymeric material and, as well, about 50% each of polycaprolactone and ethylene vinyl acetate is used.

The polymeric material used may be greater than about 99% pure and the active agents may be greater than about 97% pure. One of ordinary skill in the art will recognize that in compounding, the conditions under which compounding is carried out will need to take into account the characteristics of the active agent to ensure that the active agents do not become degraded by the process. The polycaprolactone and ethylene vinyl acetate preferably are combined with the desired active agent or agents, micro-compounded, and then extruded.

In the devices of the invention, a release-modulating component may be included. The release-modulating component may be any component that acts to modulate the release of the active agent from the plug. Suitable modulating component include, without limitation, one or more biodegradable of non-biodegradable semi-permeable membrane, one or more pores, or combinations thereof. In addition to a gradient, release of the active agent may be controlled by use of one or both of active agent loading and release enhancers or, as described on commonly-assigned, copending provisional patent application Ser. No. 61/322127, which is hereby incorporated by reference in its entirety.

In addition to or instead of active agent loading profiles, the release kinetics may be controlled via spatial gradients of the properties of degradability and drug permeability of the active agent-containing material. For example, in those cases in which drug release kinetics are dominated by the rate of material degradation, a spatial degradation in the material chemistry including, without limitation, polylactide-glycolide copolymers of differing monomer ratios, adjacent polyglycolide and polycaprolactone layers and the like, results in spatial gradients and varied release rates as the material degradation front moves through the device. By way of further example, a material may erode more slowly initially in a first, outer material and more quickly in a second, inner material to achieve phased release kinetics.

In the case of a non-degradable material that elutes the active agent solely through diffusion-dominated mechanisms, spatial gradients in the material's permeability can control release kinetics beyond what is possible with a homogeneous material. In the diffusion-dominated mechanism, the material permeability controls release kinetics and is influenced by the material's porosity as well as the active agent solubility and diffusivity. By forming an active agent-loaded layer of an outer material with a higher permeability, the active agent elution may be controlled to be more linear with less burst effect than that which is otherwise achieved with a single, homogeneous, diffusion material.

The spatial gradients in biodegradability or permeability may be combined with continuous or step-wise gradients in the active agent loading profile. For example, a punctal plug material core having an outer segment loaded with a low active agent concentration and with a relatively low active agent permeability may be adjacent to an inner material segment loaded with a high agent concentration and with a relatively high active agent permeability, which combination achieves release kinetics unobtainable with a homogeneous material ad homogeneous active agent loading. The initial burst release is reduced and the release of the last active agent content is accelerated relative to a conventional homogeneous active agent loaded device.

Phase-separated inclusions may be used to control one or both of diffusive and degradative kinetics of the active agent-containing material. For example, water soluble polymers, water soluble salts, materials with a high diffusivity for the active agent and the like may be used as destabilizing inclusion to enhance degradation or diffusion rates. When the hydrolysis front reaches an inclusion, the inclusion rapidly dissolves and increases porosity of the active agent-containing material. The inclusions may be incorporated as gradients or layers that allow additional tailoring of the release profile.

As another alternative, a percolated network of destabilizing inclusions may be used. When used in a non-biodegradable active agent-containing material, these inclusions form islands within the material that can possess high diffusivity for the active agent. Useful inclusions will have a higher diffusivity for the active agent than the active agent-containing material. Examples of such inclusions include, without limitation, propylene glycol, silicone oil, immiscible dispersed solids such as a polymer or wax and the like. As yet another example, an inclusion that acts to absorb water, swell the active agent-containing material and increase local diffusion kinetics may be used.

As still another alternative, stabilizing inclusions that have low active agent diffusivity are used. These inclusions act to form a barrier that slows diffusive transport of the active agent in the vicinity of the inclusion. The overall effect is a reduction of active agent permeability in a base material that is otherwise the same. Example of such inclusions include, without limitation, micro to nano-sized silicate particles dispersed through the base material of one or both of polycaprolactone and ethylenecovinylacetate homogeneously or in continuous step-wise gradients.

The present invention encompasses numerous devices for the delivery of active agents to the eye each having various features and advantages. For example, certain devices may have a body with a first end, a second end, and a lateral surface extending between the two ends. The lateral surface preferably has an outer diameter that is substantially circular in shape and, thus, the body preferably has a cylindrical shape. A portion of the lateral surface of certain of the devices preferably has an outer diameter that is greater than the outer diameter of the remainder of the lateral surface as shown in FIG. 1. The enlarged portion can be any size or shape, and can be present on any part of the lateral surface, in punctal plug embodiments, the enlarged portion is of a size so that it at least partially anchors the punctal plug in the lacrimal canaliculus and preferably, the enlarged portion is at one end of the plug. One ordinarily skilled in the art will recognize that any of a wide variety of shapes are possible.

The body of the punctal plugs of the invention may take any shape and size, preferably, the body is in the shape of an elongated cylinder. The body will be about 0.8 to about 5 mm in length, preferably about 1.2 to about 2.5 mm in length. The width of the body will be about 0.2 to about 3, preferably 0.3 to about 1.5 mm. The size of the opening will be from about 1 um to about 2.5 mm and preferably about 0.15 mm to about 0.8 mm. Instead of one large opening at any one location, multiple small openings may be used. The body of the plug may be wholly or partially transparent or opaque. Optionally, the body may include a tint or pigment that makes the plug easier to see when it is placed in a punctum.

The body of the devices of the invention may be made of any suitable biocompatible material including, without limitation, silicone, silicone blends, silicone co-polymers, such as, for example, hydrophilic monomers of polyhydroxyethylmethacrylate (“pHEMA”), polyethylene glycol, polyvinylpyrrolidone, and glycerol, and silicone hydrogel polymers such as, for example, those described in U.S. Pat. Nos. 5,962,548, 6,020,445, 6,099,852, 6,367,929, and 6,822,016, incorporated herein in their entireties by reference. Other suitable biocompatible materials include, for example: polyurethane; polymethylmethacrylate; poly(ethylene glycol); poly(ethylene oxide); polypropylene glycol); poly(vinyl alcohol); poly(hydroxyethyl methacrylate); poly(vinylpyrrolidone) (“PVP”); polyacrylic acid; poly(ethyloxazoline); poly(dimethyl acrylamide); phospholipids, such as, for example, phosphoryl choline derivatives; polysulfobetains; acrylic esters, polysaccharides and carbohydrates, such as, for example, hyaluronic acid, dextran, hydroxyethyl cellulose, hydroxyl propyl cellulose, gellan gum, guar gum, heparan sulfate, chondroitin sulfate, heparin, and alginate; proteins such as, for example, gelatin, collagen, albumin, and ovalbumin; polyamino acids; fluorinated polymers, such as, for example, PTFE, PVDF, and teflon; polypropylene; polyethylene; nylon; and EVA.

The surface of the devices may be wholly or partially coated. The coating may provide one or more of lubriciousness to aid insertion, muco-adhesiveness to improve tissue compatibility, and texture to aid in anchoring the device. Examples of suitable coatings include, without limitation, gelatin, collagen, hydroxyethyl methacrylate, PVP, PEG, heparin, chondroitin sulphate, hyaluronic acid, synthetic and natural proteins, and polysaccharides, thiomers, thiolated derivatives of polyacrylic acid and chitosan, polyacrylic acid, carboxymethyl cellulose and the like and combinations thereof.

Certain embodiments of the devices of the invention have a body made of a flexible material that conforms to the shape of whatever it contacts. Optionally, in the punctal plug embodiment, there may be a collarette formed of either a less flexible material than that of the body or material that too conforms to the shape of whatever it contacts. When a punctal plug having both a flexible body and a less flexible collarette is inserted into the lacrimal canaliculus, the collarette rests on the exterior of the lacrimal punctum and the body of the punctal plug conforms to the shape of the lacrimal canaliculus. The reservoir and the body of such punctal plugs are preferably coterminous. That is, the reservoir of such punctal plugs preferably comprises the entirety of the body, except for the collarette.

In embodiments in which one or both of a flexible body and collarette are used, the flexible body and flexible collarette can be made of materials that include, without limitation, nylon, polyethylene terephthalate (“PET”), polybutylene terephthalate (“PBT”), polyethylene, polyurethane, silicone, PTFE, PVDF, and polyolefins. Punctal plugs made of nylon, PET, PBT, polyethylene, PVDF, or polyolefins are typically manufactured for example and without limitation, extrusion, injection molding, or thermoforming. Punctal plugs made of latex, polyurethane, silicone, or PTFE are typically manufactured using solution-casting processes.

Processes for manufacturing the punctal plugs useful in the invention are well known. Typically, the devices are manufactured by injection molding, cast molding, transfer molding or the like. Preferably, the reservoir is filled with one or both of at least one active agent and the active agent-containing material subsequent to the manufacture of the device. Additionally, one or more excipients may be combined with the active agent alone or in combination with the polymeric material.

The amount of active agent used in the devices of the invention will depend upon the active agent or agents selected, the desired doses to be delivered via the device, the desired release rate, and the melting points of the active agent and active agent-containing material. Preferably, the amount used is a therapeutically effective amount meaning an amount effective to achieve the desired treatment, inhibitory, or prevention effect. Typically, amounts of about 0.05 to about 8,000 micrograms of active agents may be used.

In certain aspects of the invention, the reservoir can be refilled with a material after substantially all of the active agent-containing material has dissolved or degraded and the active agent is released. For example, the new active agent-containing material can be the same as, or different from, the previous polymeric material, and can contain at least one active agent that is the same as, or different from the previous active agent. Certain punctal plugs used for particular applications can preferably be refilled with a material while the punctal plugs remain inserted in the lacrimal canaliculus, while other punctal plugs are typically removed from the lacrimal canaliculus, a new material is added, and the punctal plugs are then reinserted into the lacrimal canaliculus.

In another embodiment, the invention may include discrete, parallel microchannels. Individual channels may be filled with different formulations: different drug and excipient concentration gradients, stacked layers, membrane caps, erodible and non-erodible polymers, etc. to create non-homogeneous cores with one or more sequential “pulses” from each distinct microchannel, thus enabling any combination of sustained and pulsed drug release profiles therein. The microchannels may be from about 10 to about 300 microns in diameter; may have round, rectangular, or other cross-sectional profile; and may be from about 500 to about 5000 microns long, and may have unequal lengths. The microchannels may be formed by bundling together individual tubes or channels, or formed by direct molding or etching/drilling/cutting into a single body of material.

After the device is filled with the active agent, the plug is sterilized by any convenient method including, without limitation, ethylene oxide, autoclaving, irradiation, and the like and combination thereof. Preferably, sterilization is carried out through gamma radiation or use of ethylene oxide.

The devices described herein can be used to deliver various active agents for the one or more of the treatment, inhibition, and prevention of numerous diseases and disorders. Each device may be used to deliver at least one active agent and can be used to deliver different types of active agents. For example, the devices can be used to deliver azelastine HCl, emadastine difumerate, epinastine HCl, ketotifen fumerate, levocabastine HCl, olopatadine HCl, pheniramine maleate, and antazoline phosphate for one or more of the treatment, inhibition, and prevention of allergies. The devices can be used to deliver mast cell stabilizers, such as, for example, cromolyn sodium, lodoxamide tromethamine, nedocromil sodium, and permirolast potassium.

The devices can be used to deliver mydriatics and cycloplegics including, without limitation, atropine sulfate, homatropine, scopolamine HBr, cyclopentolate HCl, tropicamide, and phenylephrine HCl. The devices can be used to deliver ophthalmic dyes including, without limitation, rose bengal, sissamine green, indocyanine green, fluorexon, and fluorescein.

The devices can be used to deliver corticosteroids including, without limitation, dexamethasone sodium phosphate, dexamethasone, fluoromethalone, fluoromethalone acetate, loteprednol etabonate, prednisolone acetate, prednisolone sodium phosphate, medrysone, rimexolone, and fluocinolone acetonide. The devices can be used to deliver non-steroidal anti-inflammatory agents including, without limitation, flurbiprofen sodium, suprofen, diclofenac sodium, ketorolac tromethamine, cyclosporine, rapamycin methotrexate, azathioprine, and bromocriptine.

The devices can be used to deliver anti-infective agents including, without limitation, tobramycin, moxifloxacin, ofloxacin, gatifloxacin, ciprofloxacin, gentamicin, sulfisoxazolone diolamine, sodium sulfacetamide, vancomycin, polymyxin B, amikacin, norfloxacin, levofloxacin, sulfisoxazole diolamine, sodium sulfacetamide tetracycline, doxycycline, dicloxacillin, cephalexin, amoxicillin/clavulante, ceftriaxone, cefixime, erythromycin, ofloxacin, azithromycin, gentamycin, sulfadiazine, and pyrimethamine.

The devices can be used to deliver agents for the one or more of the treatment, inhibition, and prevention of glaucoma including, without limitation, epinephrines, including, for example: dipivefrin; alpha-2 adrenergic receptors, including, for example, aproclonidine and brimonidine; betablockers including, without limitation, betaxolol, carteolol, levobunolol, metipranolol, and timolol; direct miotics, including, for example, carbachol and pilocarpine; cholinesterase inhibitors, including, without limitation, physostigmine and echothiophate; carbonic anhydrase inhibitors, including, for example, acetazolamide, brinzolamide, dorzolamide, and methazolamide; prostoglandins and prostamides including, without limitation, Latanoprost, bimatoprost, uravoprost, and unoprostone cidofovir.

The devices can be used to deliver antiviral agents, including, without limitation, fomivirsen sodium, foscarnet sodium, ganciclovir sodium, valganciclovir HCl, trifluridine, acyclovir, and famciclovir. The devices can be used to deliver local anesthetics, including, without limitation, tetracaine HCl, proparacaine HCl, proparacaine HCl and fluorescein sodium, benoxinate and fluorescein sodium, and benoxnate and fluorexon disodium. The devices can be used to deliver antifungal agents, including, for example, fluconazole, flucytosine, amphotericin B, itraconazole, and ketocaonazole.

The devices used to deliver analgesics including, without limitation, acetaminophen and codeine, acetaminophen and hydrocodone, acetaminophen, ketorolac, ibuprofen, and tramadol. The devices can be used to deliver vasoconstrictors including, without limitation, ephedrine hydrochloride, naphazoline hydrochloride, phenylephrine hydrochloride, tetrahydrozoline hydrochloride, and oxymetazoline. Finally, the devices can be used to deliver vitamins, antioxidants, and nutraceuticals including, without limitation, vitamins A, D, and E, lutein, taurine, glutathione, zeaxanthin, fatty acids and the like.

The active agents delivered by the devices can be formulated to contain excipients including, without limitation, synthetic and natural polymers, including, for example, polyvinylalcohol, polyethyleneglycol, PAA (polyacrylic acid), hydroxymethyl cellulose, glycerine, hypromelos, polyvinylpyrrolidone, carbopol, propyleneglycol, hydroxypropyl guar, glucam-20, hydroxypropyl cellulose, sorbitol, dextrose, polysorbate, mannitol, dextran, modified polysaccharides and gums, phosolipids, and sulphobetains.

Exemplary methods of manufacture of the drug core according to the present invention are more fully described, without limitation, in the following examples.

### EXAMPLES

Several studies have been conducted in both beagle dogs and humans that show that topically ophthalmically applying concentrated nano-doses of prostaglandins can be efficacious, and furthermore, that the dose needed for efficacy may be less than that from a standard commercially available drop, which was demonstrated by the following study results (Note: BP=bimatoprost, LP=latanoprost):

Clinical data demonstrating the potential of concentrated nanodoses to achieve efficacy is shown below in Table 1. The table shows the average change from baseline IOP data as a function of time following either ˜1 or ˜9 μg doses of bimatoprost, applied as a 20 nL volume dose. These data come from clinical studies in which ˜9 μg BP or ˜1 μg BP were dosed as 20 nL on Days 0-3. On Day 4, a single drop of Lumigan (0.03% BP, ˜30 μL was dispensed from the Lumigan dropper bottle) was administered.

These IOP (intra-ocular pressure) data were collected ˜24 hours post-dose on each day. It is important to note that the amount of bimatoprost contained in a Lumigan (0.03% BP) drop is ˜9 μg (applied as 30 μL), and daily Lumigan treatment has been shown to have a 6-8 mm Hg reduction in IOP. The Day 5 data shown in FIG. 1 below are the change from baseline IOP values following application of Lumigan. The data using ˜1 μg BP demonstrate that an IOP-lowering effect can occur using nanodoses that apply less drug than the amount contained in a standard 30-70 μL ophthalmic drop.

What is claimed is:

1. A method for treating ocular aliments, comprising:
providing a device having a quantity of concentrated active-agent containing material therein;
delivering a therapeutic effective amount of the active-agent containing material in substantially discrete doses of from about 10 nl to about 100 nl.

2. The method of claim 1 wherein the active-agent in the concentrated active-agent containing material is indicated for use to reduce intra-ocular pressure.

3. The method of claim 2 wherein the active-agent in the concentrated active-agent containing material comprises at least one of latanoprost, bimatoprost, travoprost, and mixtures thereof.

4. The method of claim 2 wherein the substantially discrete doses are delivered approximately once every 1 to about every 72 hours.

5. The method of claim 1 wherein the device comprises a punctal plug.

6. The method of claim 5 wherein the punctal plug comprises an osmotic, electric, or mechanical pumping mechanism.

7. The method of claim 5 wherein the punctal plug comprises a pumping mechanism that comprises of two or more of osmotic, electrical, or mechanical pumping mechanisms.

8. The method of claim 5 wherein the punctal plug comprises a housing having a housing having substantially parallel side walls, a first end and a second end; a collarette disposed at the first end and the second end configured to permit insertion of the device into a lacrimal punctum of from about 1 mm to about 10 mm in diameter

9. The method of claim 8 wherein the collarette is configured to rest on the ocular surface proximate to the lacrimal punctum and to inhibit the punctal plug from completely entering the lacrimal canniculus.
